(19) United States
(12) Patent Application Publication
(10) Pub. No.: US 2007/0167933 A1
(43) Pub. Date: Jul. 19, 2007

(54) Method for the control of a medical apparatus by an operator

(76) Inventor: Estelle Camus, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/541,417

(22) Filed: Sep. 29, 2006

(30) Foreign Application Priority Data
Sep. 30, 2005 (DE) 10 2005 047 044.0

(51) Int. Cl. A61B 17/00; G09B 21/00

(52) U.S. Cl. 606/1; 340/825.19

(57) ABSTRACT

In this invention a medical apparatus is controlled by capturing thought contents, wherein to this end electroencephalogram signals are recorded and the signals are evaluated with the aim of looking for certain patterns from a table or database. The method is initiated by means of a start command or ended by means of an end command. A medical apparatus according to the invention includes two types of control, namely a control via the capture of brain currents and via the capture of speech.

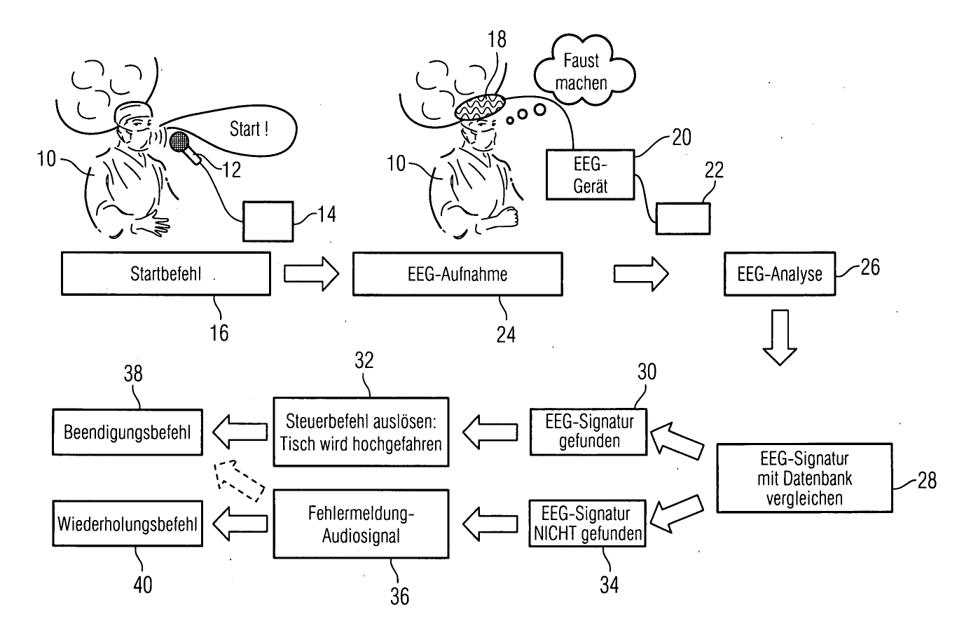

### CROSS REFERENCE TO RELATED APPLICATIONS

This application claims priority of German application No. 10 2005 047 044.0 filed Sep. 30, 2005, which is incorporated by reference herein in its entirety.

### FIELD OF THE INVENTION

The invention relates to a method for the control of a medical apparatus by an operator.

### BACKGROUND OF THE INVENTION

The medical apparatus is for example a medical apparatus for the purposes of diagnosis or therapy. During operations diagnostic devices are used in addition. The patient is lying, for example, on an X-ray table, and X-ray images are taken while the operation is being performed.

It is desirable that it is one and the same person performing the operation and operating the medical apparatus. For example, a customized selection of appropriate sections for X-ray must be made, so that the operating doctor receives optimal support during the operation. Here, the carrying out of the operation is in part in conflict with the operation of the medical apparatus. As a rule, both hands of the operating doctor are occupied with the operation on the patient. Moreover, the use of control buttons that are not necessarily sterile, are inconsistent with the hygiene required during operations.

Therefore, in the prior art, there are embodiments where foot pedals are used to control the medical apparatus. Naturally, this limits the number of functions. There are also embodiments where the operator works using speech recognition. However, the speech recognition is very susceptible to errors.

### SUMMARY OF THE INVENTION

The object of the invention is to present a method for controlling a medical apparatus, wherein one person can easily control the medical apparatus.

This object is achieved by a method and a medical apparatus that can be activated in accordance with the claims.

In the invention, a new development in the prior art is used, where the control of apparatus is initiated using brain signals. For example, Washington University in St. Louis reported in the June edition of the Journal of Neuroengineering, that electrodes attached to the cerebral membrane of patients can be used to pick up signals, with which signals the patients can control a cursor on a computer. In a development, a control of this kind is also possible using EEG signals (electroencephalogram signals).

Thus, according to the invention, EEG signals are taken from the operator. Then the operator must repeatedly think predetermined thought contents corresponding to specific EEG patterns. The predetermined thought contents can comprise the planning of specific movements that are recognizable as patterns in the EEG signals. In a next step, the pattern is extracted from the EEG steps. The extracted patterns are compared with patterns in a table, where a device command is allocated to each pattern. When an extracted pattern is found in the table, then the associated device command is executed.

The invention goes beyond the prior filed but later published application, with the official file reference 10 2005 023 033.4, in the sense that the control is initiated by means of a start command and/or ended by means of an end command.

Using the latter method, it is no longer necessary to constantly pick up and evaluate EEG signals. The method can be initiated and interrupted by start and end commands.

The start command and/or the end command can be input verbally and in the case of speech recognition they can be further processed. Thus one can use the already available prior art of speech recognition, whereby the speech recognition is used to a limited extent for the start command and for the end command and is thus less error prone than when it is used for extensive device control commands.

Alternatively, the start and/or end command can also correspond to a specific pattern in the table. In other words, the start command can also be controlled via the EEG signal by “thinking a predetermined thought content”. This is also practical in particular for the end command, as at this point in time, the EEG signals are being received continually and analyzed in any case.

The start and/or end command can also be input by means of a foot pedal. Thus the foot pedal is not totally dispensed with, but it has a relatively simple function.

The medical apparatus that can be activated according to claim 5 has, as in the application with the file number 10 2005 023 033.4, a control that comprises a device for capturing the brain currents of an operator and equipment for evaluating the captured brain currents and thus generating associated control signals for the apparatus. What is novel compared to that, is that the control also comprises a device for capturing speech, in particular a microphone, as well as equipment for evaluating the captured speech and generating associated control signals for the device for capturing brain currents or for the apparatus. Thus, with the medical apparatus according to the invention it is possible to have a combination of control commands via the brain currents with verbally input control commands. In particular, the above mentioned start and end command can be input verbally, and the other control commands can be input via brain currents. In the latter case, the equipment for evaluating the captured speech controls the device for capturing brain currents by generating associated control signals. It is, however, also possible for very specific control commands for the medical apparatus as such to be input verbally and for a different partial selection of orders to be input via the captured brain currents.

### BRIEF DESCRIPTION OF THE DRAWINGS

A preferred embodiment of the invention is described below with reference to the figure that illustrates the individual steps of the method as claimed in the invention.

### DETAILED DESCRIPTION OF THE INVENTION

The figure shows a surgeon 10, who is performing an operation on a patient (not shown), which patient is lying on an X-ray table. As a rule, the surgeon is attending to the patient. If the X-ray table with the patient were to be raised so as to be nearer to the surgeon 10, thus theoretically the surgeon 10 could manipulate a corresponding switch with his hand. Because this is, however, too unhygienic and possibly also too time consuming, the surgeon 10 inputs a start command verbally (step 16) into a microphone 12 (device for capturing speech) with a downstream speech recognition unit 14. There are electrodes 18 attached to the surgeon himself (only shown on the right in the illustration) and said electrodes are linked to an EEG apparatus 20, and together these form a device for capturing brain currents. Downstream of the EEG apparatus 20 is an evaluation unit 22, which evaluates the captured brain currents and generates associated control signals for the X-ray table. Thus in a next step 24, an electroencephalogram (EEG) is recorded. Hereby the surgeon 10 has the input requirement, that if he wants to give a specific control command, he must think specific thought contents allocated to the control command. For example, he must think that he would like to make fist (without necessarily making a fist), if he wants to move the X-ray table. As part of a subsequent electroencephalographic analysis, here as step 26, the signals picked up are examined. For example, alpha, delta and theta waves that have a certain shape are sought. The equipment 22 is accordingly designed to carry out such a customized analysis, i.e. to analyze exactly the curve parts in which the characteristic thought contents are reflected.

Several control commands are available to the surgeon, which commands are stored in a database (table), wherein, in the table one control command for the medical apparatus is allocated respectively to one thought content (“make a fist”, “lift an arm” etc.). Then in a subsequent step 28, the electroencephalogram signature captured is compared with the database. If the captured signature is found, then the equipment 22 has recognized the thought content predetermined by the surgeon in the database and can now trigger the corresponding control command: for example, the X-ray table is raised (step 32).

If, contrary to that, in the analysis after step 26, the equipment 22 is not able to find the EEG signature in the database (according to step 34), then in step 36 an error message is given, for example, an audio signal. After step 32 and, as the case may be, also after step 36, an end command 38 may be given. The surgeon 10 can also input said end command verbally into the microphone 12 (“End!”). After the error message, an order to repeat can also be issued instead (“Again!”).

With the invention it will be possible to move away from purely controlling the X-ray table through speech inputs into the microphone 12. Instead the actual main control commands are issued via the electrodes 18 and the EEG apparatus 20.

The invention profits from the fact that there are two different methods of inputting control commands available. It does not necessarily have to be the case that the verbal method of inputting a control command is only available for the start command, the end command and the repeat command; control commands could also be issued in via this route. A part of the total selection of possible control commands is, however, in every case also issued by capturing brain signals. In this way the workload of the speech input system is lightened but said system still continues to be used expediently.

The invention is not limited to the control of an X-ray table. For example, the X-ray C-arc of an X-ray angiography system could be controllable. In general, the invention is not limited to X-ray units; all kinds of medical apparatus used for therapy or diagnosis can be controlled in the ways described.

1-5. (canceled)

6. A method for controlling a medical apparatus by an operator, comprising:
taking an EEG signal from the operator;
predetermining a thought content by the operator corresponding to a specific EEG pattern;
extracting the EEG pattern from the EEG signal;
comparing the extracted pattern with a plurality of patterns in a table comprising a plurality of device commands allocated to each of the patterns;
executing an associated device command allocated to a pattern in the table identical to the extracted pattern; and
initiating the control by a start command.

7. The method as claimed in claim 6, wherein the control is ended by an end command.

8. The method as claimed in claim 7, wherein the start or end command is inputted verbally and is further processed by a speech recognition.

9. The method as claimed in claim 7, wherein the start or end commend corresponds to one of the EEG patterns in the table.

10. The method as claimed in claim 7, wherein the start or end command is inputted by a foot pedal.

11. The method as claimed in claim 6, wherein the thought content comprises a planning of a specific movement that is recognizable as the EEG pattern in the EEG signal.

12. The method as claimed in claim 6, wherein the operator repeatedly thinks the predetermined thought content.

13. A medical apparatus that can be activated with a control, comprising:
a first device which captures a brain current of an operator of the medical apparatus;
a first equipment which evaluates the captured brain current and generates a first control signal which controls the medical apparatus;
a second device which captures a speech of the operator; and
a second equipment which evaluates the captured speech and generates a second control signal which controls the first device.

14. The medical apparatus as claimed in claim 13, wherein the second control signal controls the medical apparatus.

15. The medical apparatus as claimed in claim 13, wherein the second control signal is a start or end command.

16. The medical apparatus as claimed in claim 13, wherein the second device is a microphone.
